(19) United States
(12) Patent Application Publication
(10) Pub. No.: US 2010/0248253 A1
(43) Pub. Date: Sep. 30, 2010

(54) METHOD AND KIT FOR ASSESSING RISK OF GOUT AND HYPERURICEMIA

(75) Inventors: Ying-Chin Ko, Kaohsiung City (TW); Shun-Jen Chang, Kaohsiung City (TW); Shu-Jung Wang, Kaohsiung City (TW); Shang-Lun Chiang, Kaohsiung City (TW)

Correspondence Address: VOLPE AND KOENIG, P.C., UNITED PLAZA, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 12/797,768

(22) Filed: Jun. 10, 2010

Related U.S. Application Data
(63) Continuation-in-part of application No. 12/423,424, filed on Apr. 14, 2009.

(30) Foreign Application Priority Data
Aug. 11, 2008 (TW) 097130593

(51) Int. Cl. C12Q 1/68

(52) U.S. Cl. 435/6

(57) ABSTRACT

A method for assessing a risk of suffering from a gout of a subject is provided. The method includes steps of obtaining a nucleotide sample from the subject; determining a genetic polymorphism of one of a Urate transporter 1 (URAT1) gene and an alpha-kinase 1 (ALPK1) gene in the nucleotide sample, wherein the genetic polymorphism is associated with an occurrence of the gout; and comparing the genetic polymorphism with a predetermined genetic polymorphism so as to assess the risk of suffering from the gout of the subject.

### CROSS-REFERENCE TO RELATED APPLICATIONS

This application is a Continuation-In-Part of co-pending application Ser. No. 12/423,424 filed on Apr. 14, 2009, and for which priority is claimed under 35 U.S.C. §120; and this application claims priority of Application No. 97130593 filed in Taiwan on Aug. 11, 2008 under 35 U.S.C. §119; the entire contents of all are hereby incorporated by reference.

### FIELD OF THE INVENTION

The present invention relates to a method and a kit for assessing risks of gout and hyperuricemia, and more particularly to a method and a kit for assessing risks of gout and hyperuricemia by using a single nucleotide polymorphism (SNP).

### BACKGROUND OF THE INVENTION

The homeostasis of the uric acid, which is a breakdown product of purines in mammals, depends on the balance between the production and the excretion of the uric acid. Hyperuricemia can result from either an excess production or a reduced excretion of the uric acid or a combination of both mechanisms, and in the cases of hyperuricemia, about 10-15% thereof have the symptoms of gout.

Based on the above, it could be known that gout is a metabolic disorder of urate and is characterized by the abnormal elevated level of urate in the serum of the patient. When the urate in the serum is elevated beyond the physiologic solubility limit, it will crystallize as a monosodium urate monohydrate and deposit within the joints. The Clinical severity caused by the abnormal elevated level of the urate varies from episodic to recurrent painful attacks of acute inflammatory arthritis, tophaceous gout, chronic polyarticular arthritis, and uric-acid urolithiasis, with possible sequelae of the renal impairment and failure.

In terms of the genetic regulations of the secretion and the excretion of the uric acid in the kidneys, the following molecular candidates have been proposed: OATs, URAT1, galectin-9, OATV1, and MRP4, wherein the OAT1 and the OAT3 may mediate the secretion of the urate and the URAT1 was identified as an urate transporter in the human kidney since several mutations in the N-terminus of the URAT1 gene may cause a reduced renal urate level, and identified a urate-anion exchanger for regulating a blood urate level.

Although there were studies indicating that the occurrence of gout might be associated with the genetic factors, the susceptive or pathogenic genes for gout are not entirely clear. The rare genetic mutations on rare diseases only account for a small proportion of the idiopathic hyperuricemia as well as the occurrence of the rare instances, e.g. the deficiency of hypoxanthine-guanine phosphoribosyltransferase and the over-activity of phosphorribosylpyrophosphate synthetase. It might be because of the complexity of the pathogenesis of gout, the small sample size, the nonrandom sampling, or the insufficient statistic methods, the susceptive or the pathogenic genes of gout are still unable to be identified from the genome of humans in the recent studies so far. Accordingly, up to now, it is unable to assess the risk of developing gout or hyperuricemia of an individual by detecting a specific gene, and there is no objective method which could be used to achieve the purpose as well.

Hence, because of the defects in the prior arts, the inventor identifies specific genes and single nucleotide polymorphisms (SNPs) involved therein and provides a method and a kit for assessing risks of gout and hyperuricemia to effectively overcome the demerits existing in the prior arts.

### SUMMARY OF THE INVENTION

It is an aspect of the present invention to provide a method and a kit for assessing risks of gout and hyperuricemia having the capability of assessing whether an individual has high gout and hyperuricemia risks and having an advantage of the increased reliability of the assessed result via the combination of the SNPs disclosed in the present invention. Furthermore, the method and markers disclosed in the present invention are valuable to the research of the pathogenesis and the applications of the future targeted therapy.

In accordance with an aspect of the present invention, a method for assessing a risk of suffering from a gout of a subject is provided. The method comprises steps of obtaining a nucleotide sample from the subject; determining a genetic polymorphism of one of a Urate transporter 1 (URAT1) gene and an alpha-kinase 1 (ALPK1) gene in the nucleotide sample, wherein the genetic polymorphism is associated with an occurrence of the gout; and comparing the genetic polymorphism with a predetermined genetic polymorphism so as to assess the risk of suffering from the gout of the subject.

Preferably, the subject is an alcoholic.

Preferably, the nucleotide sample is a polynucleotide sample and the genetic polymorphism is associated with a uric acid level of the subject.

Preferably, the genetic polymorphism is a single nucleotide polymorphism (SNP).

Preferably, the SNP of the URAT1 gene comprises at least one selected from a group consisting of rs505802, rs11602903, rs3825018, rs3825016, rs11231825, rs475688 and rs7932775.

Preferably, the step of determining the SNP of the URAT1 gene comprises a step of determining whether a base of the rs3825016 is a cytosine. A method as claimed in Claim 6, wherein the subject has the risk of suffering from the gout when the base of the rs3825016 is the cytosine.

Preferably, the SNP of the ALPK1 gene comprises at least one selected from a group consisting of rs916868, rs9994944, rs2074388, rs13148353, rs2074379, s11726117, rs6841595, rs11098156, rs231247, lak84, rs231253 and rs960583.

Preferably, the step of determining the SNP of the ALPK1 gene comprises a step of determining whether a base of the rs231247 is a guanine.

Preferably, the subject has the risk of suffering from the gout when the base of the rs231247 is the guanine.

In accordance with another aspect of the present invention, a method for determining a hyperuricemia susceptibility of a subject is provided. The method comprising steps of obtaining a nucleotide sample from the subject; identifying a genetic polymorphism in one of a Urate transporter 1 (URAT1) gene and an alpha-kinase 1 (ALPK1) gene in the nucleotide sample, wherein the genetic polymorphism is associated with an occurrence of the hyperuricemia; and comparing the genetic polymorphism with a predetermined genetic polymorphism so as to determine the hyperuricemia susceptibility of the subject.

Preferably, the nucleotide sample is a polynucleotide sample and the genetic polymorphism is a single nucleotide polymorphism (SNP).

Preferably, the step of identifying the SNP of the URAT1 gene comprises a step of determining whether a base of the rs3825016 is a cytosine.

Preferably, the subject has the hyperuricemia susceptibility when the base of the rs3825016 is the cytosine.

Preferably, the step of identifying the SNP of the ALPK1 gene comprises a step of determining whether a base of the rs231247 is a guanine.

Preferably, the subject has the hyperuricemia susceptibility when the base of the rs231247 is the guanine.

In accordance with a further aspect of the present invention, a kit detecting one of a Urate transporter 1-rs3825016 C marker and a alpha-kinase 1-rs231247 G marker is provided, so as to determine one of a gout susceptibility and a hyperuricemia susceptibility of a subject.

Preferably, the kit comprises one of a first reagent set for detecting the Urate transporter 1-rs3825016 C marker and a second reagent set for detecting the alpha-kinase 1-rs231247 G marker.

Preferably, the subject has one of the gout susceptibility and the hyperuricemia susceptibility when at least one of the Urate transporter 1-rs3825016 C marker and the alpha-kinase 1-rs231247 G marker is detected.

The above objects and advantages of the present invention will become more readily apparent to those ordinarily skilled in the art after reviewing the following detailed descriptions.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

Firstly, linkage analysis to fine-map GOUT1 gene within the 4q25 candidate region (114 cM˜124 cM) was performed by CEQTM 8000 Genetic Analysis System with five additional microsatellite markers derived from 21 families (92 gout cases and 62 non-gout controls). After the analysis, it is found that the maximal linkage signal moves from 114 cM to 117 cM region and at least 404 SNPs were typed in thirty-eight genes located between D4S1647 and D4S2937. Among the thirty-eight genes, four candidate genes, SCYE1, DKK2, FLJ39370 and ALPK1, comprising 75 SNPs were conducted and examined in the family and population based set of 201 gout cases and 244 controls. Since the SNPs of ALPK1 gene was found to be significantly associated with gout cases, twelve SNPs of ALPK1 gene were selected to perform the subsequent experiments. In addition, the SNPs of another two genes, OAT1 and URAT1, associated with the uric acid were used in the experiments of the present invention as well. The inventor screened 1-5 exons of URAT1 for mutations in 24 gout cases and 17 controls. There is not found any SNP in the exons 3-5 of the URAT1 gene, except 5 SNPs (rs3802948, rs12800450, rs3825017, rs3825016, and rs11231825) found in exons 1-2, which were used in the subsequent experiments of the present invention as well.

SNP Genotyping

The SNPs of ALPK1 genotyping were done by using the TaqMan SNP allelic discrimination by means of an ABI7900HT (Applied Biosystems, Foster City, Calif., USA) and that of URAT1 and OAT1 genes were identified from the public SNPs database. Twelve SNPs of ALPK1 gene selected for the present invention comprise four missense mutations: rs2074388 G565D, rs13148353H642R, rs2074379 M732I and rs11726117 M861T; two nonsense mutations: rs231247 and a novel lak84; four intron SNPs; and two 3′-Upstream Regulatory Region SNPs. Seven SNPs of URAT1 selected for the present invention comprise two 5′ near SNPs (rs505802 and rs11602903), three nonsense SNPs (rs3825016, rs11231825 and rs7932775), one 5′-Upstream Regulatory Region SNP (rs3825018) and one tag SNP (rs475688); three SNPs of OAT1 therefor comprise two tag SNPs (rs6591722 and rs2276300) and one 5′-Upstream Regulatory Region SNP (rs4149170). The SNPs of URAT1 and OAT1 were initially identified by the TaqMan in a cohort of gout subjects. Please refer to Table 1, wherein the relevant information of the 22 SNPs used in the present invention is listed therein.

TABLE 1

| Gene Symbol | SNP | Base Pair | Location |
|---|---|---|---|
| ALPK1 | rs916868 | 113566163 | intron 6 |
| (4q26) | rs9994944 | 113566945 | intron 7 |
| | rs2074388 G565D | 113571846 | exon 11 |
| | rs13148353 H642R | 113572077 | exon 11 |
| | rs2074379 M732I | 113572348 | exon 11 |
| | rs11726117 M861T | 113572734 | exon 11 |
| | rs6841595 | 113573291 | intron 11 |
| | rs11098156 | 113575944 | intron 12 |
| | rs231247 R1084R | 113579152 | exon 13 |
| | rs55840220 | 113580374 | exon 14 |
| | T1145T | | |
| | rs231253 | 113582071 | 3′-untranslated |
| | | | region |
| | rs960583 | 113582497 | 3′-untranslated |
| | | | region |
| OAT1 | rs4149170 | 62508865 | 5′-upstream |
| (11q12.3) | | | regulatory region |
| | rs6591722^{a} | 62506256 | intron 3 |
| | rs2276300^{a} | 62505275 | intron 5 |
| URAT1 | rs505802 | 64113648 | 5′ near gene |
| (11q13.1) | rs11602903 | 64114817 | 5′ near gene |
| | rs3825018 | 64115385 | 5′-upstream |
| | | | regulatory region |
| | rs3825016(H86H) | 64115862 | exon 1 |
| | rs11231825(H142H) | 64116850 | exon 2 |
| | rs475688^{a} | 64120867 | intron 4 |
| | rs7932775(L437L) | 64124438 | exon 8 |

^{a}Tag SNP variants was selected from NCBI (http://www.ncbi.nlm.nih.gov) and HapMap (http://www.hapmap.org/).

Statistical Analyses

In the present invention, the multipoint analysis is applied by using the conditional-logistic model implemented in the S.A.G.E. Version 5.4.1 program package for the linkage fine mapping analysis. The association tests as well as the other statistical analyses were done by using the Statistical Analysis Systems software version 9.1.3 (SAS Institute Inc, Cary, N.C., USA), the Haploview 4.0 and the PLINK v1.00, so as to calculate the P-value of the permutation test by using 100,000 permutations and to run the linkage disequilibrium (LD) and the haplotype association tests for the SNPs. The joint effects of ALPK1 and URAT1 genotypes and/or alcohol consumption on gout risk were evaluated on both multiplicative and additive scales. The likelihood ratio test was used to test for the interaction between the genetic traits and the environmental factors based on a multiplicative model. QUANTO 1.2.3 is used for the sample size and statistical power calculations.

Please refer to Table 2, which shows the association tests between the SNPs of ALPK1, URAT1 and OAT1 genes proposed in the present invention and the gout susceptibility in family-based and population-based 550 gout cases and 974 controls. In a combined analysis, the complete sets including 12 SNPs of ALPK1, 7 SNPs of URAT1 and 3 SNPs of OAT1 were given, and the associated risks with each of the SNPs were estimated by allelic odds ratio and the related 95% confidence intervals (CI). As shown in Table 2, in ALPK1 and URAT1 genes, the SNP rs231247 and the SNP rs3825016 has the strong main effect for gout, respectively.

The homozygosity for the allele (G) of rs231247 was associated with a significant risk of gout (odds ratio, 2.02; 95% confidence interval [CI], 1.29 to 3.18; and permutation P value=6.11×10⁻³), whereas the allelic odds ratio was 1.36 (95% CI, 1.17 to 1.59; and permutation P=8.00×10⁻⁵). In the combined analysis, the most significant SNP was found for the nonsense SNP rs3825016 of URAT1 in the homozygous CC polymorphism (odds ratio, 3.63; 95% CI, 1.44 to 9.31; and permutation P=2.80×10⁻⁴), and the allelic odds ratio was 1.48 (95% CI, 1.19 to 1.83; and permutation P=4.16×10⁻⁴). It is also shown in Table 2 that the three SNPs of OAT1 gene did not significantly associate with the gout disease.

TABLE 2

| Num- | | Risk | Gout | Control | Gout | Control | Allelic | p for | | |
|---|---|---|---|---|---|---|---|---|---|---|
| ber | SNP | allele | Frequency | Frequency | AA/Aa/aa | AA/Aa/aa | OR (95% CI)^{c} | Genotype | p for Allele | p for Trend |
| | ALPK1 gene | | | | | | | | | |
| 1 | rs916868 | C | 0.68 | 0.65 | 180/173/38 | 213/226/64 | 1.16 (0.95-1.42) | 3.00 × 10⁻¹ | 1.44 × 10⁻¹ | 1.45 × 10⁻¹ |
| 2 | rs9994944 | G | 0.67 | 0.64 | 245/230/62 | 391/430/133 | 1.17 (1.00-1.37) | 1.63 × 10⁻¹ | 5.53 × 10⁻² | 5.82 × 10⁻² |
| 3 | rs2074388(D > G) | A | 0.59 | 0.53 | 188/261/92 | 274/451/222 | 1.28 (1.10-1.49) | 5.01 × 10⁻³ | 1.35 × 10⁻³ | 1.46 × 10⁻³ |
| 4 | rs13148353(R > H) | G | 0.61 | 0.56 | 199/255/83 | 305/434/192 | 1.22 (1.04-1.42) | 3.38 × 10⁻² | 1.33 × 10⁻² | 1.49 × 10⁻² |
| 5 | rs2074379(I > M) | A | 0.61 | 0.55 | 146/182/61 | 161/230/112 | 1.28 (1.06-1.55) | 3.21 × 10⁻² | 1.03 × 10⁻² | 1.24 × 10⁻² |
| 6 | rs11726117(T > M) | C | 0.62 | 0.55 | 205/246/80 | 313/424/212 | 1.30 (1.12-1.52) | 2.15 × 10⁻³ | 7.50 × 10⁻⁴ | 1.35 × 10⁻³ |
| 7 | rs6841595 | C | 0.63 | 0.56 | 158/174/59 | 170/228/106 | 1.30 (1.07-1.57) | 3.23 × 10⁻² | 7.64 × 10⁻³ | 1.05 × 10⁻² |
| 8 | rs11098156 | T | 0.20 | 0.16 | 25/153/323 | 22/131/393 | 1.33 (1.06-1.66) | 3.29 × 10⁻² | 1.21 × 10⁻² | 1.64 × 10⁻² |
| 9 | rs231247(R > R) | G | 0.62 | 0.54 | 208/238/83 | 288/453/207 | 1.36 (1.17-1.59) | 4.60 × 10⁻⁴ | 8.00 × 10⁻⁵ | 9.00 × 10⁻⁵ |
| 10 | rs55840220(T > T)^{b} | A | 0.18 | 0.11 | 25/140/373 | 30/140/779 | 1.82 (1.47-2.26) | 3.32 × 10⁻⁸ | 8.67 × 10⁻⁹ | 9.51 × 10⁻⁸ |
| 11 | rs231253 | G | 0.61 | 0.54 | 196/243/85 | 275/431/203 | 1.31 (1.12-1.53) | 3.30 × 10⁻³ | 7.50 × 10⁻⁴ | 8.90 × 10⁻⁴ |
| 12 | rs960583 | A | 0.21 | 0.17 | 27/167/333 | 27/251/630 | 1.31 (1.08-1.59) | 1.91 × 10⁻² | 6.01 × 10⁻³ | 6.74 × 10⁻³ |
| | URAT1 gene | | | | | | | | | |
| 13 | rs505802 | C | 0.89 | 0.85 | 275/59/5 | 284/101/9 | 1.57 (1.14-2.15) | 1.63 × 10⁻² | 4.93 × 10⁻³ | 5.46 × 10⁻³ |
| 14 | rs11602903 | A | 0.83 | 0.77 | 245/69/24 | 254/113/34 | 1.39 (1.07-1.80) | 2.73 × 10⁻² | 1.20 × 10⁻² | 2.38 × 10⁻² |
| 15 | rs3825018 | G | 0.90 | 0.84 | 271/57/6 | 278/105/12 | 1.69 (1.24-2.32) | 3.54 × 10⁻³ | 8.62 × 10⁻⁴ | 1.17 × 10⁻³ |
| 16 | rs3825016(H > H) | C | 0.88 | 0.83 | 407/121/6 | 642/278/25 | 1.48 (1.19-1.83) | 1.62 × 10⁻³ | 4.16 × 10⁻⁴ | 3.50 × 10⁻⁴ |
| 17 | rs11231825(H > H) | T | 0.87 | 0.84 | 405/118/8 | 660/261/23 | 1.34 (1.08-1.67) | 2.74 × 10⁻² | 7.73 × 10⁻³ | 7.42 × 10⁻³ |
| 18 | rs475688 | C | 0.65 | 0.64 | 217/258/61 | 377/450/121 | 1.05 (0.90-1.22) | 7.37 × 10⁻¹ | 5.67 × 10⁻¹ | 5.61 × 10⁻¹ |
| 19 | rs7932775(L > L) | C | 0.62 | 0.61 | 156/173/65 | 303/420/120 | 1.03 (0.87-1.22) | 1.46 × 10⁻¹ | 7.41 × 10⁻¹ | 7.40 × 10⁻¹ |
| | OAT1 gene | | | | | | | | | |
| 20 | rs2276300 | A | 0.26 | 0.22 | 16/81/120 | 13/102/181 | 1.28 (0.95-1.71) | 2.31 × 10⁻¹ | 1.00 × 10⁻¹ | 1.03 × 10⁻¹ |
| 21 | rs6591722 | T | 0.83 | 0.80 | 151/61/6 | 194/87/15 | 1.22 (0.89-1.69) | 3.74 × 10⁻¹ | 2.23 × 10⁻¹ | 2.35 × 10⁻¹ |
| 22 | rs4149170 | A | 0.26 | 0.23 | 27/113/182 | 20/129/224 | 1.20 (0.93-1.53) | 2.60 × 10⁻¹ | 1.68 × 10⁻¹ | 1.78 × 10⁻¹ |

Please refer to Table 3, which shows the association test between the SNPs of ALPK1, URAT1 and OAT1 genes proposed in the present invention and the risk of hyperuricemia. Table 3 is completed by analyzing 12 SNPs of ALPK1, 7 SNPs of URAT1 and 3 SNPs of OAT1 in 981 hyperuricemia cases and 543 normal uric acid level controls, wherein the P value was analysed after 100,000 permutations using PLINK v1.00 software. Hyperuricemia was defined as the serum uric acid over 7 mg/dL for males and over 6 mg/dL for females. The result of the association tests in this Table is similar to that in Table 2 and also reveals that the SNP rs231247 of ALPK1 or the SNP rs3825016 of URAT1 has the strong main effects for the risk of hyperuricemia.

TABLE 3

| | | | | Minor | | | Major | Chi- | |
|---|---|---|---|---|---|---|---|---|---|
| Number | CHR | SNP | Base Pair | Allele | Hyperuricemia | Controls | Allele | Square | p-Value |
| | | ALPK1 gene | | | | | | | |
| 1 | 4 | rs916868 | 113566163 | T | 0.33 | 0.37 | C | 2.18 | 0.364 |
| 2 | 4 | rs9994944 | 113566945 | A | 0.34 | 0.38 | G | 3.03 | 0.058 |
| 3 | 4 | rs2074388(D > G) | 113571846 | G | 0.44 | 0.49 | A | 5.61 | 0.018 |
| 4 | 4 | rs13148353(R > H) | 113572077 | A | 0.41 | 0.45 | G | 4.14 | 0.049 |
| 5 | 4 | rs2074379(I > M) | 113572348 | G | 0.41 | 0.48 | A | 7.27 | 0.011 |
| 6 | 4 | rs11726117(T > M) | 113572734 | T | 0.42 | 0.44 | C | 1.88 | 0.172 |
| 7 | 4 | rs6841595 | 113573291 | A | 0.39 | 0.46 | C | 5.85 | 0.021 |
| 8 | 4 | rs11098156 | 113575944 | T | 0.19 | 0.15 | G | 3.75 | 0.101 |
| 9 | 4 | rs231247(R > R) | 113579152 | A | 0.42 | 0.45 | G | 2.42 | 0.121 |
| 10 | 4 | rs55840220(T > T) | 113580374 | A | 0.14 | 0.11 | G | 3.30 | 0.113 |
| 11 | 4 | rs231253 | 113582071 | C | 0.42 | 0.47 | G | 4.37 | 0.042 |
| 12 | 4 | rs960583 | 113582497 | A | 0.19 | 0.17 | G | 1.37 | 0.286 |
| | | URAT1 gene | | | | | | | |
| 13 | 11 | rs505802 | 64113648 | T | 0.13 | 0.11 | C | 1.03 | 0.333 |
| 14 | 11 | rs11602903 | 64114817 | T | 0.19 | 0.22 | A | 1.21 | 0.393 |
| 15 | 11 | rs3825018 | 64115385 | A | 0.14 | 0.14 | G | 0.00 | 0.857 |
| 16 | 11 | rs3825016(H > H) | 64115862 | T | 0.15 | 0.18 | C | 6.66 | 0.020 |
| 17 | 11 | rs11231825(H > H) | 64116850 | C | 0.14 | 0.17 | T | 2.47 | 0.171 |
| 18 | 11 | rs475688 | 64120867 | T | 0.36 | 0.37 | C | 0.06 | 0.808 |
| 19 | 11 | rs7932775(L > L) | 64124438 | T | 0.38 | 0.41 | C | 2.59 | 0.123 |
| | | OAT1 gene | | | | | | | |
| 20 | 11 | rs2276300 | 62505275 | A | 0.24 | 0.24 | G | 0.00 | 0.981 |
| 21 | 11 | rs6591722 | 62506256 | A | 0.18 | 0.20 | T | 0.66 | 0.427 |
| 22 | 11 | rs4149170 | 62508865 | A | 0.24 | 0.23 | G | 0.11 | 0.765 |

Please refer to Table 4, which shows the correlations between the odds ratios of the gout risk and several confounding factors including the age, the gender, the familial aggregation, and the alcohol use. In this Table, the symbol “OR” representing the odds ratio without considering the confounding factors is a result of a univariate analysis, and the symbol “aOR (adjusted odds ratio)” representing the odds ratio after adjusting the confounding factors is a result of a multivariate analysis. Theoretically, since the result of a multivariate analysis more conforms to the actual situations, the reliability thereof is higher than that of a univariate analysis. Based on Table 4, either of the univariate analysis and the multivariate analysis indicates the significant interaction between the confounding factors, the gender (male) and the alcohol use, and the gout risk. Particularly, heavy drinkers including long-time drinkers and drinkers having an alcohol intake over 45 g/day are significantly related with the gout occurrence. Furthermore, Table 4 also shows significant interactions between the rs231247 G allele of ALPK1 and the increasing risk of gout (odds ratio, 1.36; and 95% CI, 1.14 to 1.64) and between the rs3825016 C allele of URAT1 and that of gout (odds ratio, 1.56; and 95% CI, 1.21 to 2.01).

Please refer to Table 5, which shows the independent and the synergistic interaction between the SNPs of ALPK1 and URAT1 genes proposed in the present invention and the confounding factor of the alcohol consumption. Evidence of a gene-environment interaction was found between the genotypes of the SNP rs231247 of ALPK1 or the SNP rs3825016 of URAT1 and the alcohol consumption. The observed joint risk for heavy alcoholics (over 45 g/day of drinking) who carried the at-risk G allele of rs231247 of ALPK1 (odds ratio, 5.03; and 95% CI, 3.70 to 6.82) or the at-risk C allele of rs3825016 of URAT1 (odds ratio, 8.53; and 95% CI, 5.22 to 13.94) was significantly higher than the expected risk estimated from a multiplicative model. In addition, a significant additive interaction between the combination of the rs231247 G and the rs3825016 C alleles and the alcohol intake was found with an increasing risk of gout (odds ratio, 10.26; and 95% CI, 3.93 to 26.83). Accordingly, the risk-predicted result obtained by using the SNPs of ALPK1 and URAT1 genes simultaneously as the genetic markers is more reliable than that obtained by independently using the SNP of one of the two genes as the genetic maker. Since the alcohol intake increases the production of the lactate, which is a substrate of URAT1, the lactate reabsorption is coupled with the increased urate reabsorption or competitively inhibits the secretion of the renal urate, thereby reducing the excretion of the renal urate. The “alcohol associated gout” is caused by the dual effect of either the overproduction or the under-excretion of the urate, which may enhance the occurrence of hyperuricemia or gout.

TABLE 4

| | Gout | Control | | |
|---|---|---|---|---|
| Variable | (n = 550) | (n = 974) | OR (95% CI) | aOR (95% CI)^{c} |
| Age (years) | 51.0 (14.5) | 53.9 (17.2) | 0.99 (0.98-1.00) | 1.02 (1.01-1.02) |
| Gender (%) | | | | |
| Female | 126 (23) | 479 (49) | 1.00 | 1.00 |
| Male | 424 (77) | 495 (51) | 3.26 (2.57-4.12) | 1.79 (1.45-2.21) |
| Study group (%) | | | | |
| Population-based | 458 (83) | 912 (94) | 1.00 | 1.00 |
| Family-based | 92 (17) | 62 (6) | 2.95 (2.10-4.15) | 3.53 (2.62-4.76) |
| Alcohol use (%)^{b} | | | | |
| Never Drinker | 110 (20) | 446 (46) | 1.00 | 1.00 |
| Drinker | 440 (80) | 528 (54) | 3.38 (2.65-4.31) | — |
| 1-10 (years) | 46 (8) | 96 (10) | 1.94 (1.29-2.92) | — |
| 11-20 (years) | 95 (17) | 138 (14) | 2.79 (2.00-3.90) | — |
| >20 (years) | 299 (54) | 294 (30) | 4.12 (3.17-5.37) | — |
| Intake (%) | | | | |
| 1-45 (g/day) | 78 (14) | 193 (20) | 1.64 (1.17-2.29) | 1.26 (0.95-1.66) |
| >45 (g/day) | 362 (66) | 335 (34) | 4.38 (3.39-5.66) | 3.42 (2.74-4.27) |
| SNPs | | | | |
| ALPK1, rs231247 (%) | | | | |
| A | 38 | 46 | 1.00 | 1.00 |
| G | 62 | 54 | 1.36 (1.17-1.59) | 1.36 (1.14-1.64) |
| URAT1, rs3825016 (%) | | | | |
| T | 12 | 17 | 1.00 | 1.00 |
| C | 88 | 83 | 1.48 (1.19-1.83) | 1.56 (1.21-2.01) |
| Biologic | | | | |
| Characteristics^{a} | | | | |
| Total cholesterol (mg/dl) | 187.2 (48.6) | 183.7 (48.0) | 1.00 (1.00-1.00) | |
| Triglyceride (mg/dl) | 274.0 (279.8) | 204.2 (278.4) | 1.00 (1.00-1.00) | |
| Log Triglyceride | 5.3 (0.8) | 5.0 (0.8) | 1.78 (1.54-2.05) | 1.33 (1.18-1.50) |
| Creatinine (mg/dl) | 1.2 (0.4) | 1.0 (0.5) | 3.34 (2.23-5.00) | 1.19 (0.99-1.44) |
| Uric acid (mg/dl) | 9.3 (2.4) | 7.2 (2.0) | 1.54 (1.45-1.63) | 1.39 (1.33-1.45) |

^{a}Value expressed as mean (standard deviation) unless otherwise.
^{b}Collinearity in alcohol use items, the adjusted odds ratios are presented only in the alcohol intake.
^{c}Odds ratio (OR) were adjusted for SNPs, alcohol intake, other covariates in the table, and the associated 95% confidence intervals (CI).

TABLE 5

| Gene/ | Nondrinker | | 1-45 g/day of Drinking | | >45 g/day of Drinking | |
|---|---|---|---|---|---|---|
| Allele | Gout/Control | aOR (95% CI) | Gout/Control | aOR (95% CI) | Gout/Control | aOR (95% CI) |
| ALPK1 | | | | | | |
| rs231247 | | | | | | |
| A | 80/393 | 1.00 | 54/180 | 1.37 (0.91-2.07) | 270/292 | 4.02 (2.92-5.53) |
| G | 130/471 | 1.20 (0.86-1.66) | 92/196 | 2.17 (1.50-3.14) | 430/358 | 5.03 (3.70-6.82) |
| URAT1 | | | | | | |
| rs3825016 | | | | | | |
| T | 22/163 | 1.00 | 15/49 | 2.44 (1.14-5.25) | 96/116 | 5.87 (3.37-10.22) |
| C | 192/701 | 2.21 (1.34-3.64) | 135/323 | 3.31 (1.97-5.55) | 608/538 | 8.53 (5.22-13.94) |
| rs231247/ | | | | | | |
| rs3825016 | | | | | | |
| A/T | 5/47 | 1.00 | 2/8 | 2.03 (0.32-12.90) | 16/32 | 4.17 (1.34-12.94) |
| G/T | 17/115 | 1.14 (0.39-3.39) | 13/41 | 2.79 (0.89-8.79) | 80/82 | 7.31 (2.69-19.88) |
| A/C | 73/340 | 2.00 (0.75-5.32) | 52/172 | 2.59 (0.96-7.04) | 253/258 | 8.03 (3.06-21.05) |
| | 113/352 | 2.66 (1.01-7.01) | 77/151 | 4.39 (1.63-11.79) | 345/270 | 10.26 (3.93-26.83) |

Please refer to Table 6, which shows the genetic association analysis of the genotypes of URAT1 gene from different groups. In this embodiment, the subjects from Han Chinese and the subjects from Solomon Islanders are taken as examples for the above-mentioned different groups. As shown, two of 4 SNPs shows significant associations with gout across the 2 groups (genotypic p<0.05). The individual risk for having gout with the rs475688 CC genotype (at-risk homozygote) is higher in Han Chinese, and substantially replicated in Solomon Islanders, adjusted for correlated covariates, and compared to wild-type homozygote controls (OR=3.88, 95% CI=1.66-9.09 and OR=4.12, 95% CI=1.38-12.31, respectively). Table 6 also indicates that an at-risk allele of SNP rs475688 is significantly association with gout, with the allelic p=0.008 (adjusted OR=1.89, 95% CI=1.28-2.80) as well as at-risk allele frequency of 0.51 in the controls and 0.62 in the affected individuals in Han Chinese, and with allelic p=0.012 (adjusted OR=1.81, 95% CI=1.12-2.90) as well as at-risk allele frequency of 0.45 in the controls and 0.59 in the affected individuals among Solomon Islanders. Both risk allele frequencies are similar in cases from samples of geographically diverse populations. SNP rs7932775 is also significantly association with gout (CC vs. TT, adjusted OR=2.57, 95% CI=1.20-5.50), with allelic p=0.012 (adjusted OR=1.65, 95% CI=1.12-2.42) as well as a risk allele frequency of 0.46 in the controls and 0.56 in the affected individuals in Han Chinese.

Please refer to Table 7, which shows the genetic association analysis of the genotypes of ALPK1 gene from different groups. The mentioned different groups are the same as those for Table 6. In this embodiment, the tophaceous gout patients are evaluated separately, and the results indicate that SNP rs475688 is significantly associated with this phenotype in Han Chinese (CC vs. TT, OR=3.98, 95% CI=1.01-15.72; allelic OR=1.85, 95% CI=1.00-3.41). For Solomon Islanders with this phenotype, there is a significant shift to rs7932775 L437L (allelic OR=2.72, 95% CI=1.33-5.56). In conclusion, the results in Tables 6 and 7 indicate no matter whether the samples have the commonness of specific populations, either the SNP of ALPK1 or the SNP of URAT1 is significantly associated with the risk of gout.

TABLE 6
Genetic association for SNPs of URAT1 gene with gout in two independent groups*

| | Risk/ | | | | | | |
|---|---|---|---|---|---|---|---|
| | Reference | Gout | | Control | | Genotypic | Allelic |
| SNP | Allele | AA/Aa/aa | RAF | AA/Aa/aa | RAF | p-Value | p-Value |
| Han | | | | | | | |
| Chinese | | | | | | | |
| rs7932775, | C/T | 36/56/22 | 0.56 | 51/81/65 | 0.46 | 0.035 | 0.020 |
| exon 8 | | | | | | | |
| rs475688, | C/T | 44/52/17 | 0.62 | 49/106/45 | 0.51 | 0.020 | 0.008 |
| intron 4 | | | | | | | |
| rs11231825, | T/C | 70/38/7 | 0.77 | 133/57/12 | 0.80 | 0.652 | 0.447 |
| exon 2 | | | | | | | |
| rs3825016, | C/T | 62/51/2 | 0.76 | 118/81/3 | 0.78 | 0.737 | 0.490 |
| exon 1 | | | | | | | |
| Solomon | | | | | | | |
| Islanders | | | | | | | |
| rs7932775, | C/T | 2/31/20 | 0.33 | 9/53/75 | 0.26 | 0.047 | 0.166 |
| exon 8 | | | | | | | |
| rs475688, | C/T | 16/32/6 | 0.59 | 28/68/42 | 0.45 | 0.018 | 0.012 |
| intron 4 | | | | | | | |
| rs11231825, | T/C | 16/31/7 | 0.58 | 62/59/17 | 0.66 | 0.134 | 0.143 |
| exon 2 | | | | | | | |
| rs3825016, | C/T | 16/31/7 | 0.58 | 60/61/17 | 0.66 | 0.192 | 0.185 |
| exon 1 | | | | | | | |

| | p for | aOR (95% CI)† | aOR (95% CI) | aOR (95% CI) |
|---|---|---|---|---|
| SNP | HWE | (Homozygous) | (Heterozygous) | (Allelic) |
| Han | | | | |
| Chinese | | | | |
| rs7932775, | 0.015 | 2.57 (1.20-5.50) | 2.37 (1.19-4.71) | 1.65 (1.12-2.42) |
| exon 8 | | | | |
| rs475688, | 0.393 | 3.88 (1.66-9.09) | 2.12 (0.96-4.67) | 1.89 (1.28-2.80) |
| intron 4 | | | | |
| rs11231825, | 0.089 | 1.07 (0.34-3.38) | 1.24 (0.38-4.12) | 0.94 (0.59-1.50) |
| exon 2 | | | | |
| rs3825016, | 0.008 | 0.38 (0.05-2.84) | 0.40 (0.05-2.99) | 0.91 (0.58-1.42) |
| exon 1 | | | | |
| Solomon | | | | |
| Islanders | | | | |
| rs7932775, | 0.930 | 0.92 (0.17-5.02) | 2.72 (1.32-5.59) | 1.57 (0.94-2.64) |
| exon 8 | | | | |
| rs475688, | 0.960 | 4.12 (1.38-12.31) | 3.01 (1.12-8.10) | 1.81 (1.12-2.90) |
| intron 4 | | | | |
| rs11231825, | 0.612 | 0.59 (0.20-1.78) | 1.26 (0.44-3.55) | 0.69 (0.43-1.12) |
| exon 2 | | | | |
| rs3825016, | 0.806 | 0.60 (0.20-1.81) | 1.24 (0.44-3.5) | 0.71 (0.44-1.14) |
| exon 1 | | | | |

*‘A’ denotes risk allele, ‘a’ denotes non-risk allele;
RAF denotes risk allele frequency;
HWE: Hardy-Weinberg Equilibrium.
†Odds ratio (OR) for a SNP was adjusted for age, total cholesterol, log-transformed triglyceride, creatinine and alcohol drink (yes/no) compared to controls with wild-type counterparts.

TABLE 7
Genetic association for SNPs of URAT1 in male with tophaceous gout in two independent groups*

| | | Tophaceous Gout | | Control | | | | |
|---|---|---|---|---|---|---|---|---|
| | Risk/ | Genotypic | | Genotypic | | | | |
| | Reference | Frequency | | Frequency | | aOR (95% CI)† | aOR (95% CI) | aOR (95% CI) |
| SNP | Allele | (AA/Aa/aa)* | Allele | (AA/Aa/aa) | Allele | (Homozygous) | (Heterozygous) | (Allelic) |
| Han Chinese | | | | | | | | |
| rs7932775 | C/T | 0.29/0.54/0.17 | 46/36 | 0.26/0.41/0.33 | 183/211 | 2.38 (0.70-8.08) | 2.42 (0.83-7.06) | 1.55 (0.86-2.80) |
| rs475688 | C/T | 0.37/0.49/0.15 | 50/32 | 0.25/0.53/0.23 | 204/196 | 3.98 (1.01-15.72) | 2.33 (0.66-8.29) | 1.85 (1.00-3.41) |
| rs11231825 | T/C | 0.54/0.42/0.05 | 61/21 | 0.66/0.28/0.06 | 323/81 | 0.76 (0.13-4.40) | 1.25 (0.21-7.42) | 0.72 (0.37-1.43) |
| rs3825016 | C/T | 0.51/0.49/0 | 62/20 | 0.58/0.40/0.01 | 317/87 | — | — | 0.95 (0.48-1.89) |
| Solomon | | | | | | | | |
| Islanders | | | | | | | | |
| rs7932775 | C/T | 0.09/0.73/0.18 | 20/24 | 0.07/0.39/0.55 | 71/203 | 4.74 (0.69-32.83) | 6.95 (2.03-23.84) | 2.72 (1.33-5.56) |
| rs475688 | C/T | 0.27/0.59/0.14 | 25/19 | 0.20/0.49/0.30 | 124/152 | 4.04 (0.85-19.29) | 2.10 (0.52-8.50) | 1.80 (0.90-3.61) |
| rs11231825 | T/C | 0.36/0.55/0.09 | 28/16 | 0.45/0.43/0.12 | 183/93 | 0.72 (0.13-4.14) | 1.21 (0.22-6.72) | 0.76 (0.37-1.56) |
| rs3825016 | C/T | 0.36/0.55/0.09 | 28/16 | 0.43/0.44/0.12 | 181/95 | 0.73 (0.13-4.20) | 1.19 (0.22-6.62) | 0.77 (0.38-1.59) |

*‘A’ denotes risk allele, ‘a’ denotes non-risk allele.
†Odds ratio (OR) for a SNP was adjusted for age, total cholesterol, log-transformed triglyceride, creatinine and alcohol drink (yes/no) compared to controls with wild-type counterparts.

Please refer to Table 8, which shows the associations between the uric acid levels and the gene-gene interactions, which is analyzed by the genotypes of the SNPs rs231247 and rs3825016 provided in the present invention. In order to investigate the role of the gene-gene interaction in determining the uric acid levels, the multilocus genotype in the multivariate models is tested in the present invention, in which both the age and the gender were included as the confounding factors. As shown in Table 8, the at-risk G allele of the rs231247 (the uric acid levels of G and A alleles are 8.35±0.09 mg/dl versus 8.08±0.11 mg/dl, P=0.040) and the C allele of the rs3825016 (the uric acid levels of C and T alleles are 8.32±0.08 versus 7.90±0.16 mg/dl, P=0.016) were associated with higher serum uric acid levels and independently increased risk for gout. In addition, the present invention also discloses that the at-risk G-C was associated with higher serum uric acid levels (the uric acid levels of the G-C and the A-T combinations are 8.40±0.10 mg/dl versus 7.23±0.32 mg/dl, P=0.003) and evidences for the presence of the gene-gene interactions on increasing risk for gout (odds ratio, 3.99; and 95% CI, 2.06 to 7.72).

TABLE 8

| | Serum Uric | | |
|---|---|---|---|
| | Acid (mg/dl) | | |
| | Mean ± SE | P value | aOR (95% CI) |
| rs231247_ALPK1 | | | |
| A (N = 542) | 8.08 ± 0.11 | | 1.00 |
| G (N = 868) | 8.35 ± 0.09 | 0.040 | 1.47 (1.18-1.84) |
| rs3825016_URAT1 | | | |
| T (N = 222) | 7.90 ± 0.16 | | |
| C (N = 1186) | 8.32 ± 0.08 | 0.016 | 2.02 (1.48-2.75) |
| rs231247-rs3825016 | | | |
| A-T | 7.23 ± 0.32 | | 1.00 |
| G-T | 8.14 ± 0.19 | | 2.20 (1.07-4.50) |
| A-C | 8.19 ± 0.11 | | 2.84 (1.45-5.54) |
| G-C | 8.40 ± 0.10 | 0.003 | 3.99 (2.06-7.72) |

Please refer to Table 9, which shows the associations of the risk haplotype analysis of the gout risk across the three missense SNPs, rs2074388, rs13148353, and rs11726117, and the nonsense SNP rs231247 of ALPK1 gene, and the two nonsense SNPs, rs3825016 and rs11231825, of URAT1 gene. The results of Table 9 are obtained from 550 gout cases and 974 controls. Regarding ALPK1 gene, as shown in Table 9, the at risk haplotype (AGCG) analysis indicates that four SNPs (rs2074388 A>G, rs13148353 G>A, rs11726117 C>T and rs231247 G>A) of ALPK1 remained significant after 100,000 permutation analysis (P=8.00×10⁻⁴). The occurrence frequency of the ALPK1 AGCG haplotype is 0.56 among the case subjects and 0.49 among the control subjects, respectively, and that of the ALPK1 GATA haplotype is 0.35 among the case subjects and 0.39 among the control subjects, respectively. The Haplotype analysis showed that the odds ratio for the gout disease with the ALPK1 AGCG haplotype, as compared with the GATA haplotype, was 1.29 (95% CI, 1.10 to 1.51) per copy of the haplotype. Regarding URAT1 gene, as shown in Table 9, the at risk haplotype (CT) analysis indicates that two nonsense SNPs (rs3825016 T<C and rs11231825 C<T) of URAT1 remained significant after 100,000 permutation analysis (P=2.00×10⁻⁵). The occurrence frequency of the URAT1 CT haplotype (rs3825016 C and rs11231825 T) is 0.86 among the case subjects and 0.78 among the control subjects, respectively, and that of the URAT1 TC haplotype is 0.11 among the case subjects and 0.15 among the control subjects, respectively. The Haplotype analysis showed that the odds ratio for the gout disease with the URAT1 CT haplotype, as compared with the TC haplotype, was 1.46 (95% CI, 1.14 to 1.87) per copy of the haplotype.

TABLE 9

| | | | | | |
|---|---|---|---|---|---|
| | Gout | Controls | Chi- | | |
| Haplotype | n (%) | n (%) | Square | p-Value^{b} | OR (95% CI) |

| | | | | | |
|---|---|---|---|---|---|
| ALPK1 | | | | | |
| rs2074388/rs13148353/ | | | | | |
| rs11726117/rs231247 | | | | | |
| AGCG | 605 (56) | 939 (49) | 15.30 | 8.00 × 10⁻⁴ | 1.29 (1.10-1.51) |
| GGCG | 22 (2) | 38 (2) | 0.29 | 0.999 | 1.29 (0.77-2.14) |
| GACA | 22 (2) | 38 (2) | 0.01 | 1.000 | 1.07 (0.61-1.90) |
| GGTA | 4 (0.4) | 38 (2) | 14.00 | 1.50 × 10⁻³ | 0.19 (0.07-0.54) |
| AGTG | 11 (1) | 19 (1) | 0.36 | 0.998 | 0.91 (0.44-1.87) |
| GATG | 11 (1) | 19 (1) | 0.00 | 1.000 | 1.13 (0.55-2.32) |
| GATA | 378 (35) | 747 (39) | 4.92 | 0.196 | 1.00 |
| URAT1 | | | | | |
| rs3825016/rs11231825 | | | | | |
| CT | 924 (86) | 939 (78) | 23.21 | 2.00 × 10⁻⁵ | 1.46 (1.14-1.87) |
| TT | 21 (2) | 48 (4) | 11.89 | 0.002 | 0.56 (0.32-0.99) |
| CC | 11 (1) | 36 (3) | 6.92 | 0.029 | 0.61 (0.32-1.19) |
| TC | 118 (11) | 181 (15) | 6.90 | 0.029 | 1.00 |

Please refer to Table 10, which shows the associations of the risk haplotype analysis of the hyperuricemia risk across the six SNPs, rs505802, rs11602903, rs3825018, rs3825016, rs11231825 and rs7932775, of URAT1 gene. The results of Table 10 are obtained from 565 hyperuricemia cases and 151 control subjects. As shown in Table 10, the risk haplotype (CAGCTC) analysis of URAT1 for hyperuricemia indicates that six SNPs (rs505802 T<C, rs11602903 T<A, rs3825018 A<G, rs3825016 T<C, rs11231825 C<T and rs7932775 C<T) remained significant after 100,000 permutation analysis (P=0.002).

TABLE 10

| | | | Chi | |
|---|---|---|---|---|
| Haplotype | Cases (%) | Controls (%) | Square | P-value |
| CAGCTC | 55 | 45 | 9.38 | 2.20 × 10⁻³ |
| CAGCTT | 25 | 29 | 2.00 | 1.58 × 10⁻¹ |
| TTATCT | 10 | 8 | 1.68 | 1.95 × 10⁻¹ |

Based on the foregoing embodiments, it could be known that the variations found in the two genes URAT1 and ALPK1 would cause the increases in risks of suffering from gout and hyperuricemia under the independent or the synergistic effect. Therefore, the concept of the present invention is to effectively predict or assess the risks of suffering from gout and hyperuricemia by detecting the variations presenting in the two genes URAT1 and ALPK1, which could be used to classify subgroups as well. In addition, the present invention could be applied to the development of new drugs or biological products for gout and hyperuricemia, or applied to the research of the pathogenesis pathway involved in the occurrence of gout or hyperuricemia. Said method of detecting the variations in the two genes URAT1 and ALPK1 is achieved by detecting any one of the disclosed SNPs significantly associated with risk of gout or hyperuricemia, and the process of detecting any one of the disclosed SNPs could be performed by kits, packaged products, reagents or the like that is well-known in this field.

While the invention has been described in terms of what is presently considered to be the most practical and preferred embodiments, it is to be understood that the invention needs not be limited to the disclose embodiments. Therefore, it is intended to cover various modifications and similar arrangements included within the spirit and scope of the appended claims, which are to be accorded with the broadest interpretation so as to encompass all such modifications and similar structures.

What is claimed is:

1. A method for assessing a risk of suffering from a gout of a subject, comprising steps of:
obtaining a nucleotide sample from the subject;
determining a genetic polymorphism of one of a Urate transporter 1 (URAT1) gene and an alpha-kinase 1 (ALPK1) gene in the nucleotide sample, wherein the genetic polymorphism is associated with an occurrence of the gout; and
comparing the genetic polymorphism with a predetermined genetic polymorphism so as to assess the risk of suffering from the gout of the subject.

2. A method as claimed in claim 1, wherein the subject is an alcoholic.

3. A method as claimed in claim 1, wherein the nucleotide sample is a polynucleotide sample and the genetic polymorphism is associated with a uric acid level of the subject.

4. A method as claimed in claim 1, wherein the genetic polymorphism is a single nucleotide polymorphism (SNP).

5. A method as claimed in claim 4, wherein the SNP of the URAT1 gene comprises at least one selected from a group consisting of rs505802, rs11602903, rs3825018, rs3825016, rs11231825, rs475688 and rs7932775.

6. A method as claimed in claim 5, wherein the step of determining the SNP of the URAT1 gene comprises a step of determining whether a base of the rs3825016 is a cytosine.

7. A method as claimed in claim 6, wherein the subject has the risk of suffering from the gout when the base of the rs3825016 is the cytosine.

8. A method as claimed in claim 4, wherein the SNP of the ALPK1 gene comprises at least one selected from a group consisting of rs916868, rs9994944, rs2074388, rs13148353, rs2074379, s11726117, rs6841595, rs11098156, rs231247, lak84, rs231253 and rs960583.

9. A method as claimed in claim 8, wherein the step of determining the SNP of the ALPK1 gene comprises a step of determining whether a base of the rs231247 is a guanine.

10. A method as claimed in claim 9, wherein the subject has the risk of suffering from the gout when the base of the rs231247 is the guanine.

11. A method for determining a hyperuricemia susceptibility of a subject, the method comprising steps of:
obtaining a nucleotide sample from the subject;
identifying a genetic polymorphism in one of a Urate transporter 1 (URAT1) gene and an alpha-kinase 1 (ALPK1) gene in the nucleotide sample, wherein the genetic polymorphism is associated with an occurrence of the hyperuricemia; and
comparing the genetic polymorphism with a predetermined genetic polymorphism so as to determine the hyperuricemia susceptibility of the subject.

12. A method as claimed in claim 11, wherein the nucleotide sample is a polynucleotide sample and the genetic polymorphism is a single nucleotide polymorphism (SNP).

13. A method as claimed in claim 12, wherein the SNP of the URAT1 gene comprises at least one selected from a group consisting of rs505802, rs11602903, rs3825018, rs3825016, rs11231825, rs475688 and rs7932775.

14. A method as claimed in claim 13, wherein the step of identifying the SNP of the URAT1 gene comprises a step of determining whether a base of the rs3825016 is a cytosine.

15. A method as claimed in claim 12, wherein the SNP of the ALPK1 gene comprises at least one selected from a group consisting of rs916868, rs9994944, rs2074388, rs13148353, rs2074379, s11726117, rs6841595, rs11098156, rs231247, lak84, rs231253 and rs960583.

16. A method as claimed in claim 15, wherein the step of identifying the SNP of the ALPK1 gene comprises a step of determining whether a base of the rs231247 is a guanine.

17. A method as claimed in claim 16, wherein the subject has the hyperuricemia susceptibility when the base of the rs231247 is the guanine.

18. A kit detecting one of a Urate transporter 1-rs3825016 C marker and a alpha-kinase 1-rs231247 G marker, so as to determine one of a gout susceptibility and a hyperuricemia susceptibility of a subject.

19. A kit as claimed in claim 18, comprising one of:
a first reagent set for detecting the Urate transporter 1-rs3825016 C marker; and
a second reagent set for detecting the alpha-kinase 1-rs231247 G marker.

20. A kit as claimed in claim 18, wherein the subject has one of the gout susceptibility and the hyperuricemia susceptibility when at least one of the Urate transporter 1-rs3825016 C marker and the alpha-kinase 1-rs231247 G marker is detected.
